(19) United States
(12) Patent Application Publication
(10) Pub. No.: US 2020/0171088 A1
(43) Pub. Date: Jun. 4, 2020

(54) TREATMENT OF LIVER FAILURE WITH ACTIVATED T REGULATORY CELLS

(71) Applicant: Viera Bioscience, Inc., Rancho Santa Fe, CA (US)

(72) Inventor: Thomas E. Ichim, San Diego, CA (US)

(21) Appl. No.: 16/624,009

(22) PCT Filed: Jun. 20, 2018
(86) PCT No.: PCT/US2018/038481

§ 371 Date: Dec. 18, 2019

Related U.S. Application Data
(60) Provisional application No. 62/522,321, filed on Jun. 20, 2017.

(51) Int. Cl. A61K 35/17; C12N 5/0783; A61P 1/16

(52) U.S. Cl. CPC A61K 35/17; A61K 9/0019; A61P 1/16; C12N 2501/2304; C12N 2501/231; C12N 2501/2313; C12N 2501/232; C12N 2501/15; C12N 2501/21; C12N 2501/165; C12N 2501/31; C12N 2501/02; C12N 2502/02; C12N 2502/1157; C12N 2502/1107; C12N 2502/1164; C12N 2502/1121; C12N 2502/1114; C12N 5/0637

(57) ABSTRACT

Systems, compositions, and methods for the treatment of a liver disorder is disclosed. The systems, compositions, and methods include use of activated T regulatory cells for alleviating, treating, or reducing a liver disorder. The T regulatory cells may be allogeneic T regulatory cells that may be present in an amount of about 5×10⁵ to 2×10⁶ cells. The liver disorder needing treatment may be hepatitis, cirrhosis, chronic liver disease, acute liver disease, or liver failure.

### BACKGROUND

### Field of the Disclosure

The application pertains to systems, compositions, and methods for the treatment of liver failure. More particularly, the systems, compositions, and methods augment liver regenerative processes. Additionally, the systems, methods, and compositions include use of T regulatory cells that have been endowed with immune modulatory/angiogenic properties to stimulate liver regeneration while at the same time reducing liver fibrosis.

### Description of the Related Art

Liver failure is a major burden on our health care system and the seventh largest cause of death in industrialized countries. To date the only cure for liver failure is transplantation, which is severely limited by lack of donors and adverse effects of chronic immune suppression. Liver failure is caused because of a number of acute and chronic clinical inciting factors, including drug/alcohol-induced hepatotoxicity, viral infections, vascular injury, autoimmune disease, or genetic predisposition. Kelso, L. A., Cirrhosis: caring for patients with end-stage liver failure. Nurse Pract, 2008, 33(7): p. 24-30; quiz 30-1. Manifestations of liver failure include fulminant acute hepatitis, chronic hepatitis, or cirrhosis. Subsequent to various acute insults to the liver, the organ regenerates due to its unique self-renewal activity. If the insult is continuously occurring, the liver's capacity to regenerate new cells is overwhelmed and fibrotic non-functional tissue is deposited which takes over the function of the hepatic parenchyma. The subsequent reduction of hepatocyte function can give rise to metabolic instability combined with disruption of essential bodily functions (i.e., energy supply, acid-base balance and coagulation). Bernuau, J., B. Rueff, and J. P. Benhamou, Fulminant and subfulminant liver failure: definitions and causes. Semin Liver Dis, 1986, 6(2): p. 97-106; Farci, P., et al., Hepatitis C virus-associated fulminant hepatic failure. N Engl J Med, 1996, 335(9): p. 631-4; Navarro, V. J. and J. R. Senior, Drug-related hepatotoxicity. N Engl J Med, 2006, 354(7): p. 731-9. If not rapidly addressed, complications of hepatic dysfunction such as uncontrolled bleeding and sepsis occur, and dependent organs such as the brain and kidneys cease to function because of accumulation of toxic metabolites. Sargent, S., Management of patients with advanced liver cirrhosis. Nurs Stand, 2006, 21(11): p. 48-56; quiz 58. In critical cases, such as when patients progress to Acute-to-Chronic Live Failure (ACLF), liver transplant is considered the standard treatment. However, there are often serious difficulties to obtain a suitable donor and many complications arise after transplantation, including rejection and long-term adherence to immunosuppressant regimes. Kisseleva, T., E. Gigante, and D. A. Brenner, Recent advances in liver stem cell therapy. Curr Opin Gastroenterol, 2010, 26(4): p. 395-402; Wu, Y. M., et al., Hepatocyte transplantation and drug-induced perturbations in liver cell compartments. Hepatology, 2008, 47(1): p. 279-87. Although stem cell therapies are currently in development for treatment of liver failure, these possess numerous shortcomings. Embryonic and iPS derived stem cells are all difficult to grow in large quantities and possess the possibility of carcinogenesis or teratoma formation. Additionally, ectopic tissue differentiation in the hepatic microenvironment could have devastating consequences. Adult stem cells offer the possibility of inducing some clinical benefit; however, responses to date have not been profound. This is in part because of the inability of adult stem cells to fully take over hepatic tissue.

### SUMMARY

The present disclosure relates to immune modulation induced by administration of expanded T regulatory cells as a means of inducing regeneration of injured liver and inhibiting liver failure. More specifically, it is an aspect of this disclosure to provide improved systems, compositions, and methods for liver treatment. Embodiments provided herein relate to systems, compositions, and methods for the treatment of a liver disorder.

Some embodiments relate to a pharmaceutical composition for treating a liver disorder. In some embodiments, the pharmaceutical composition includes activated T regulatory cells. In some embodiments, the composition is formulated for administration to a subject having a liver disorder. In some embodiments, the activated T regulatory cells are allogeneic T regulatory cells. In some embodiments, the activated T regulatory cells produce hepatocyte growth factor. In some embodiments, the activated T regulatory cells enhance hepatic oval cell production. In some embodiments, the composition is formulated for intravenous administration. In some embodiments, the activated T regulatory cells are present in an amount of about 5×10⁵ to 2×10⁶ cells. In some embodiments, the activated T regulatory cells are present in an amount of about 1×10⁶ cells per milliliter. In some embodiments, the composition decreases a subject's serum alanine aminotransferase (ALT), aspartate aminotransferase (AST), albumin, or bilirubin levels to 5-20%, 10-30%, 20-40%, 30-50%, 40-60%, or 50-70% that of a healthy subject. In some embodiments, the liver disorder is hepatitis, cirrhosis, chronic liver disease, acute liver disease, or liver failure. In some embodiments, the composition further includes a compound for treating liver disease. In some embodiments, the composition further includes a pharmaceutically acceptable carrier.

Some embodiments relate to an infusion system, including a bag and a delivery device. In some embodiments, the bag includes a pharmaceutical composition for treating a liver disorder. In some embodiments, the pharmaceutical composition includes activated T regulatory cells. In some embodiments, the pharmaceutical composition is formulated for administration to a subject having a liver disorder. In some embodiments, the activated T regulatory cells are allogeneic T regulatory cells. In some embodiments, the activated T regulatory cells produce hepatocyte growth factor. In some embodiments, the activated T regulatory cells enhance hepatic oval cell production. In some embodiments, the composition is formulated for intravenous administration. In some embodiments, the activated T regulatory cells are present in an amount of about 5×10⁵ to 2×10⁶ cells. In some embodiments, the activated T regulatory cells are present in an amount of about 1×10⁶ cells per milliliter. In some embodiments, the composition decreases a subject's serum alanine aminotransferase (ALT), aspartate aminotransferase (AST), albumin, or bilirubin levels to 5-20%, 10-30%, 20-40%, 30-50%, 40-60%, or 50-70% that of a healthy subject. In some embodiments, the liver disorder is hepatitis, cirrhosis, chronic liver disease, acute liver disease, or liver failure. In some embodiments, the pharmaceutical composition further comprises a compound for treating liver disease. In some embodiments, the pharmaceutical composition further includes a pharmaceutically acceptable carrier.

Some embodiments relate to a method of treating liver failure. In some embodiments, the method includes administering a pharmaceutical composition as described herein. In some embodiments, the pharmaceutical composition includes activated T regulatory cells. In some embodiments, the pharmaceutical composition is formulated for administration to a subject having a liver disorder. In some embodiments, the activated T regulatory cells are allogeneic T regulatory cells. In some embodiments, the activated T regulatory cells produce hepatocyte growth factor. In some embodiments, the activated T regulatory cells enhance hepatic oval cell production. In some embodiments, the composition is formulated for intravenous administration. In some embodiments, the activated T regulatory cells are present in an amount of about 5×10⁵ to 2×10⁶ cells. In some embodiments, the activated T regulatory cells are present in an amount of about 1×10⁶ cells per milliliter. In some embodiments, the composition decreases a subject's serum alanine aminotransferase (ALT), aspartate aminotransferase (AST), albumin, or bilirubin levels to 5-20%, 10-30%, 20-40%, 30-50%, 40-60%, or 50-70% that of a healthy subject. In some embodiments, the liver disorder is hepatitis, cirrhosis, chronic liver disease, acute liver disease, or liver failure. In some embodiments, the pharmaceutical composition further comprises a compound for treating liver disease. In some embodiments, the pharmaceutical composition further includes a pharmaceutically acceptable carrier.

In some embodiments, the composition includes a population of T regulatory cells activated to possess enhanced production of liver regenerating factors by culture with allogeneic mesenchymal stem cells. In some embodiments, the population of T regulatory cells is or has been rendered activated to induce liver proliferation by enhanced ability to produce hepatocyte growth factor, or to enhance hepatic oval cell proliferation. In some embodiments, the T regulatory cells are treated with an immune modulator prior to administration.

In some embodiments, the culture with said immune modulator induces T regulatory cells proliferation or induces T regulatory cell production of leukemia inhibitory factor. In some embodiments, the immune modulator is IL-4, IL-10, IL-13, IL-20, TGF-beta, CXCL12, VEGF, PGE-2, or inhibin, or a combination thereof. In some embodiments, the T regulatory cells are cocultured with type 2 monocytes, CD5 positive B cells, type 2 NKT cells, tolerogenic dendritic cells, gamma delta T cells, T cells with immune regulatory properties, CD34 cells, very small embryonic like stem cells, or Sertoli cells.

In some embodiments, the mesenchymal stem cells are in a mitotically inactivated state. In some embodiments, the mesenchymal stem cells are cultured with an immune modulator prior to administration. In some embodiments, the mesenchymal stem cells are derived from Wharton's Jelly, bone marrow, peripheral blood, mobilized peripheral blood, endometrium, hair follicle, deciduous tooth, testicle, adipose tissue, skin, amniotic fluid, cord blood, omentum, muscle, amniotic membrane, periventricular fluid, placental tissue, pluripotent stem cells, embryonic stem cells, inducible pluripotent stem cells, parthenogenic stem cells, or somatic cell nuclear transfer derived stem cells. In some embodiments, the mesenchymal stem cells express STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, or THY-1, or a combination thereof. In some embodiments, the mesenchymal stem cells do not express substantial levels of HLA-DR, CD117, and CD45.

In some embodiments, the embryonic stem cells express stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, or human telomerase reverse transcriptase (hTERT). In some embodiments, the inducible pluripotent stem cells express CD10, CD13, CD44, CD73, CD90, PDGFr-alpha, PD-L2, or HLA-A,B,C. In some embodiments, the inducible pluripotent stem cells undergo at least 40 doublings in culture, while maintaining a normal karyotype upon passaging. In some embodiments, the parthenogenic stem cells are generated by addition of a calcium flux inducing agent to activate an oocyte followed by enrichment of cells expressing SSEA-4, TRA 1-60, orTRA 1-81. In some embodiments, the somatic cell nuclear transfer derived stem cells possess a phenotype negative for SSEA-1 and positive for SSEA-3, SSEA-4, TRA-1-60, TRA-1-81, or alkaline phosphatase.

In some embodiments, the mesenchymal stem cells are differentiated from a pluripotent stem cell source through culture in the presence of an inhibitor of the SMAD-2/3 pathway or an inhibitor nucleic acid targeting the SMAD-2/3 pathway. In some embodiments, the inhibitor is SB-431542, an antisense oligonucleotide, a hairpin loop short interfering RNA, a chemically synthesized short interfering RNA molecule, or a hammerhead ribozyme. In some embodiments, the mesenchymal stem cells inhibit hepatic stellate cell activation, inhibit hepatic fibrosis, stimulate hepatic regeneration, or augment hepatic oval cell or liver progenitor cell activity.

In some embodiments, treating liver failure comprises reducing liver fibrosis. In some embodiments, treating liver failure comprises stimulating proliferation of liver tissue growth after injury. In some embodiments, the T regulatory cells are derived from peripheral blood mononuclear cells, mobilized peripheral blood mononuclear cells, cord blood, menstrual blood, or adipose stromal vascular fraction cells. In some embodiments, the mobilized peripheral blood mononuclear cells are obtained from administration of G-CSF, flt-3 ligand, thrombopoietin, or Mozobil. In some embodiments, the activated T regulatory cells inhibit proliferation of naive T cells stimulated with a signal that activates proliferation. In some embodiments, the signal that activates proliferation is anti-CD3 and anti-CD28 beads, concanavalin A, PHA, or stimulation with alloreactive antigen presentation cells. In some embodiments, the activated T regulatory cells suppress maturation of dendritic cells. In some embodiments, the activated T regulatory cells express neuropilin-1, CTLA-4, CD25, CD39, CD73, CD105, CD127, FoxP3, GARP, GITR ligand, IL-10, or membrane bound TGF-beta. In some embodiments, the activated T regulatory cells are activated by exposure to vasoactive intestinal peptide, IL-10, TGF-beta, mesenchymal stem cell conditioned media, mesenchymal stem cell derived exosomes, BDNF, human chorionic gonadotropin, VEGF, CD3 or CD28 antibodies, hypoxic conditions, rapamycin, or angiopoietin. In some embodiments, the activated T regulatory cells are anergic T cells.

### BRIEF DESCRIPTION OF THE DRAWINGS

FIG. 1 schematically illustrates an example infusion system according to the present disclosure.

FIG. 2 graphically depicts results of reduction of alanine aminotransferase (ALT) in serum of subjects treated with a pharmaceutical composition according to the present disclosure.

### DETAILED DESCRIPTION

Before the present compositions and methods are described, it is to be understood that they are not limited to the particular compositions, methodologies or protocols described, as these may vary. It is also to be understood that the terminology used in the description is for describing the particular versions or embodiments only, and is not intended to limit their scope, which will be limited only by the appended claims.

As used herein and in the appended claims, the singular forms “a,” “an,” and “the” include plural reference unless the context clearly dictates otherwise. Unless defined otherwise, all technical and scientific terms used herein have the same meanings as commonly understood by one of ordinary skill in the art. Although any methods and materials similar or equivalent to those described herein can be used in the practice or testing of embodiments disclosed, the preferred methods, devices, and materials are now described.

The terms “patient,” “subject,” “individual,” and the like are used interchangeably herein, and refer to any animal, including mammals. In certain non-limiting embodiments, the patient, subject or individual is a human.

A “disease” is a state of health of a subject wherein the subject cannot maintain homeostasis, and wherein if the disease is not ameliorated, the subject's health continues to deteriorate. A “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health. As used herein, “treating a disease or disorder” means reducing the frequency and/or severity with which a sign or symptom of the disease or disorder is experienced by an individual.

The term “treat,” as used herein, means reducing the frequency and/or severity of a sign or symptom of a disease or disorder experienced by a subject. Thus, “treat” and “treating” are not limited to the case where the subject (e.g., patient) is cured and the disease or disorder is eradicated. Rather, the present disclosure also contemplates treatment that merely reduces a sign or symptom, improves (to some degree) and/or delays disease or disorder progression. The term “treatment” also refers to the alleviation, amelioration, and/or stabilization of signs or symptoms, as well as a delay in the progression of signs or symptoms of a disease or disorder. As used herein, to “alleviate” a disease or disorder means to reduce the frequency and/or severity of one or more signs and/or symptoms of the disease or disorder experienced by the subject.

The term “effective amount”, as used herein, refers to an amount that provides a therapeutic or prophylactic benefit in the subject. The term “therapeutically effective amount” refers to the amount of the compound that will elicit a biological or medical response of a tissue, system, animal or human that is being sought by the researcher, veterinarian, medical doctor or other clinician. The term “therapeutically effective amount” includes that amount of a compound that, when administered, is sufficient to prevent development of, or alleviate to some extent, one or more of the signs and/or symptoms of the disease or disorder being treated. The therapeutically effective amount will vary depending on the compound, the disease or disorder, the severity of the disease or disorder, and the age, weight, etc., of the subject to be treated.

### Pharmaceutical Compositions

Embodiments provided herein relate to pharmaceutical compositions for treating a liver disorder. In some embodiments, the pharmaceutical composition is provided or introduced into an infusion system. In some embodiments, the pharmaceutical composition includes activated T regulatory cells. In some embodiments, the pharmaceutical composition includes activated allogeneic T regulatory cells. In some embodiments, the activated T regulatory cells produce hepatocyte growth factor. In some embodiments, the activated T regulatory cells enhance hepatic oval cell production. In some embodiments, the activated T regulatory cells are present in an amount of about 5×10⁵ to 2×10⁶ cells. In some embodiments, the activated T regulatory cells are present in an amount of about 1×10⁶ cells per milliliter. In some embodiments, the composition decreases a subject's serum alanine aminotransferase (ALT), aspartate aminotransferase (AST), albumin, or bilirubin levels to 5-20%, 10-30%, 20-40%, 30-50%, 40-60%, or 50-70% that of a healthy subject. In some embodiments, the liver disorder is hepatitis, cirrhosis, chronic liver disease, acute liver disease, or liver failure. In some embodiments, the pharmaceutical composition further includes a compound for treating liver disease.

In some embodiments, the composition is formulated for administration to a subject having a liver disorder. In some embodiments, the composition is formulated for parenteral administration, including, for example, intravenous, subcutaneous, intraperitoneal, intramuscular, intrastemal injection, or bolus injections.

The term “pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

In some embodiments, the pharmaceutical composition further includes a pharmaceutically acceptable carrier. As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes all coatings, antibacterial and antifungal agents, absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound or molecule useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof. As used herein, the term “composition” refers to a mixture of at least one compound or molecule useful within the disclosure with one or more different compound, molecule, or material. As used herein “pharmaceutical composition” or “pharmaceutically acceptable composition” refers to specific examples of compositions, wherein at least one compound or molecule useful within the disclosure is mixed with one or more pharmaceutically acceptable carriers. In some instances, the pharmaceutical composition facilitates administration of the compound or molecule to a patient.

### Infusion Systems

The present disclosure relates to infusion systems including a pharmaceutical composition for the treatment of a liver disorder. In some embodiments, the infusion system includes a container that holds a pharmaceutical composition and a delivery device.

In some embodiments, the container is a bag, a syringe, a flask, or any suitable container for holding a pharmaceutical composition for subsequent dispensing. The container can be any container suitable for storing the composition. In some embodiments, the container can be, for example, a pre-filled syringe, a pre-filled cartridge, a vial, an ampule, or the like. In other embodiments, the container can be a container having a flexible wall, such as, for example, a bladder. In some embodiments, the container maintains sterility. In some embodiments, the container includes one or more ports, including for example, a medication port, an administration port, an inlet port, an outlet port. In some embodiments, the administration port is an outlet port through which a pharmaceutical composition may flow. In some embodiments, the administration port is fluidly connected to a tube through which a pharmaceutical composition may flow. As described herein, the tube may be any suitable tube, and may be of standard size, dimension, and material suitable for delivery of a pharmaceutical composition. A pharmaceutical composition flows from the container through the tube to a delivery device.

In some embodiments, the delivery device is a needle, a syringe, or a cannula. The delivery device enables delivery of the pharmaceutical composition through parenteral administration. As used herein, parenteral administration of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrastemal injection, or bolus injections.

In some embodiments, the infusion system further includes a drip chamber, a back check valve, an injection port, a slide clamp, a roller clamp, a threaded lock, a cap, a filter, or other components for regulating and controlling the flow of a pharmaceutical composition through the delivery device to a subject. Furthermore, the infusion systems described herein may include features enabling automated delivery of the pharmaceutical compositions, and thus may further include pumps, actuators, valves, circuits, or computer processors and memory.

In some embodiments, the infusion system can be configured to automatically deliver compositions described herein. In some embodiments, the infusion system, after being actuated by the user, can automatically produce a force to deliver the composition. In this manner, the force with which the composition is delivered is within a desired range, and is repeatable between different devices, users or the like. In some embodiments, the infusion system includes an energy storage member configured to produce a force to deliver the composition.

In some embodiments, the energy storage member can be any suitable device or mechanism that, when actuated, produces a force to deliver the composition. In some embodiments, the energy storage member can be any suitable device or mechanism that produces the force such that the composition is conveyed from the container into a body of a patient. The composition be conveyed into a body parenterally through the delivery device. In some embodiments, employing the energy storage member to produce the force, rather than relying on a user to manually produce the delivery force, the composition can be delivered to the body at a desired pressure and/or flow rate. In some embodiments, the energy storage member reduces likelihood of partial delivery of the pharmaceutical composition.

In some embodiments, the energy storage member can be a mechanical energy storage member, such as a spring, a device containing compressed gas, a device containing a vapor pressure-based propellant or the like. In other embodiments, the energy storage member can be an electrical energy storage member, such as a battery, a capacitor, a magnetic energy storage member or the like. In yet other embodiments, the energy storage member can be a chemical energy storage member, such as a container containing two substances that, when mixed, react to produce energy.

In some embodiments, the energy storage member can be in any position and/or orientation relative to the container. In some embodiments, for example, the energy storage member can be positioned within a housing spaced apart from the container. Moreover, in some embodiments, the energy storage member can be positioned such that a longitudinal axis of the energy storage member is offset from the container. In other embodiments, the energy storage member can substantially surround the container.

In some embodiments, the energy storage member can be operably coupled to the container and/or the composition contained therein such that a force delivers the composition. In some embodiments, for example, the force can be transmitted to the composition via a piston or plunger. In other embodiments, the force can be transmitted to the composition via a hydraulic or pneumatic coupling. In yet other embodiments, the force can be transmitted to the composition electrically. In still other embodiments, the force can be transmitted to the composition via a combination of any of the above.

In some embodiments, a container can include an elastomeric member, such that the force produced by an energy storage member is transferred to the composition by the elastomeric member. In some embodiments, the infusion system includes a housing, a container, an elastomeric member and an energy storage member. The container is disposed within the housing, and contains a composition. The composition can be any of the compositions described herein. The energy storage member is disposed within the housing, and is configured to produce a force to deliver the composition, as described herein.

In some embodiments, the infusion system can be any suitable device for automatically delivering any of the compositions described herein. In some embodiments, the infusion system can be a medical injector configured to automatically deliver a composition. In some embodiments, the medical injector includes a housing, a delivery mechanism, a container containing a pharmaceutical composition for treating a liver disorder, a cover, a safety lock, and/or a system actuator assembly. The medical injector may be similar to the auto-injectors described in U.S. Pat. No. 7,648,482, entitled “Devices, Systems and Methods for Medicament Delivery,” filed Nov. 21, 2006, which is incorporated herein by reference in its entirety.

In some embodiments, the delivery device is coupled to the container and defines, at least in part, a flow path through which the composition can be delivered into a body. In some embodiments, the delivery device can be directly coupled to a distal end portion of the container. In other embodiments, the delivery device can be indirectly coupled to the container.

In some embodiments, the delivery device can be coupled to, but fluidically isolated from, the container prior to actuation of the energy storage member. In this manner, the infusion system can be stored for extended periods of time while maintaining the sterility of the composition contained within the container, reducing (or eliminating) any leakage of the composition from the container. This arrangement also reduces and/or eliminates the assembly operations before the infusion system can be used to administer the composition. In this manner, the infusion system produces a quick and accurate mechanism for delivering the composition. Reducing and/or eliminating the assembly operations prior to use reduces likelihood that performance of the infusion system and/or the delivery device will be compromised (e.g., by an improper coupling, a leak or the like).

In some embodiments, the delivery device can be coupled to the container via a coupling member. In such an embodiment, the container and/or the delivery device can be configured to move relative to the coupling member when the energy storage member is actuated. Such movement can fluidically couple the delivery device and the container, thereby defining a flow path through which the composition can be delivered to the patient.

In some embodiments, the delivery device can enhance the delivery of the composition thereby improving the efficacy of the composition. In some embodiments, the delivery device can produce a flow of the composition having desired characteristics to enhance the absorption rate of the composition, to minimize the delivery of the composition to regions of the body in which such delivery is less effective, or the like.

For example, in some embodiments, the delivery device can produce a controlled flow rate of the composition. In such embodiments, the delivery device can include one or more flow orifices, a tortuous flow path or the like, to produce a desired pressure drop and/or to control the flow through the delivery device. For example, in some embodiments, the delivery device can be configured to minimize excessive delivery of the composition.

In some embodiments, for example, the delivery device and the energy storage member can be cooperatively configured such that, when the energy storage member is actuated, the infusion system produces an amount of composition within a therapeutically effective range. In some embodiments, the energy storage member is configured to “match” the delivery device such that the energy storage member is configured to produce a force within a predetermined range to ensure the desired functionality of the delivery device. Accordingly, the energy storage member can be any suitable device or mechanism that, when actuated, produces the desired force to deliver the composition as described herein. By employing the energy storage member to produce the force, rather than relying on a user to manually produce the delivery force, the composition can be delivered into the body at the desired pressure and/or flow rate.

In some embodiments, the delivery device can be coupled to, but fluidically isolated from, the container prior to actuation of the container (e.g., by manually depressing a plunger, squeezing a trigger, or the like).

In some embodiments, the delivery device can be coupled to the container via a coupling member. In such an embodiment, the container and/or the delivery device can be configured to move relative to the coupling member when the container is actuated. For example, in use, upon depressing a plunger to actuate the container, the coupling member can move relative to the container before a substantial portion of the energy produced by movement of the plunger is exerted on the composition. Such movement can fluidically couple the delivery device and the container, thereby defining a flow path through which the composition can be delivered to the patient.

The containers and/or infusion systems disclosed herein can contain any suitable amount of any of the pharmaceutical compositions for treating a liver disorder disclosed herein. For example, in some embodiments, a infusion system can be a single-dose device containing a single dosage amount of the composition to be delivered. In other embodiments, the infusion system can be a multi-dose device containing multiple dosage amounts of the composition to be delivered.

Although various embodiments have been described as having particular features and/or combinations of components, other embodiments are possible having a combination of any features and/or components from any of embodiments where appropriate. Any of the containers described herein can contain any of the compositions and/or formulations described herein.

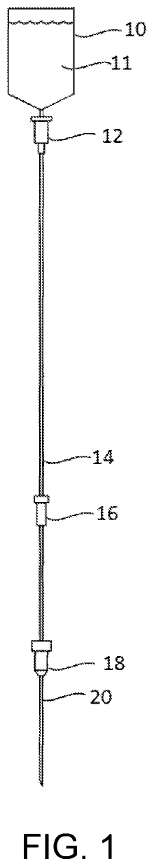

FIG. 1 depicts an example infusion system that includes a container 10 having pharmaceutical composition 11 therein. The system also includes a delivery device 20, depicted in FIG. 1 as a needle. The example system shown in FIG. 1 also includes a drip chamber 12, tubing 14, a roller clamp 16, and a threaded lock 18 that integrates with the needle 20. It is to be understood that the system shown in FIG. 1 is exemplary, and that various iterations or modifications may be realized within the scope of the application.

In some embodiments, the pharmaceutical composition is pre-packed within the container, and the container is attached to the delivery device. In some embodiments, a container includes a neutral composition, such as saline, to which a pharmaceutical composition is introduced, for example in an injection port. The pharmaceutical composition is described elsewhere herein in detail.

### T Regulatory Cells

The systems and compositions described herein include an activated T regulatory cell. Accordingly, some embodiments, provided herein relate to T regulatory cells that have been primed in vitro by culture with mesenchymal stem cells, or immune modulatory means, are provided to accelerate the process of normal liver regeneration, or to protect the process of normal liver regeneration from fibrosis. It has been demonstrated that up to 70% resection of the liver results in complete regeneration. Fausto, N., J. S. Campbell, and K. J. Riehle, Liver regeneration. Hepatology, 2006, 43(2 Suppl 1): p. S45-53; Michalopoulos, G. K., Liver regeneration: alternative epithelial pathways. Int J Biochem Cell Biol, 2011, 43(2): p. 173-9. However, this is in situations where there is no inhibition of hepatocyte proliferation. In these situations, the liver depends on proliferation of oval cells. In one embodiment of the disclosure, T regulatory cells are utilized to stimulate oval cell proliferation while concurrently inhibiting stellate cell activation.

In one aspect, T regulatory cells are administered in order to allow patients to undergo procedures such as living donor transplantation, two-stage hepatectomies, and split liver transplantation, which would be impossible for patients with various liver pathologies or fibrosis. Clavien, P. A., et al., Strategies for safer liver surgery and partial liver transplantation. N Engl J Med, 2007, 356(15): p. 1545-59; Adam, R., et al., Two-stage hepatectomy: A planned strategy to treat irresectable liver tumors. Ann Surg, 2000, 232(6): p. 777-85; Brown, R. S., Jr., Live donors in liver transplantation. Gastroenterology, 2008, 134(6): p. 1802-13; Michalopoulos, G. K., Principles of liver regeneration and growth homeostasis. Compr Physiol, 2013, 3(1): p. 485-513; Van Thiel, D. H., et al., Rapid growth of an intact human liver transplanted into a recipient larger than the donor. Gastroenterology, 1987, 93(6): p. 1414-9.

There are three phases to liver regeneration that the disclosure teaches an intervention can be made through the use of fibroblasts that have been pre-activated or “primed”: a) Priming; b) Proliferation and c) Termination. Fausto, N., J. S. Campbell, and K. J. Riehle, Liver regeneration. Hepatology, 2006, 43(2 Suppl 1): p. S45-53. It is important to note that hepatocytes are not terminally differentiated cells, but cells that reside in a state of proliferative quiescence. Specifically, they share features with other regenerative cells such hematopoietic stem cells, in that they are normally in the GO phase of cell cycle. This is altered during liver regeneration, which is described below. Without being bound to theory, the disclosure teaches that administration of T regulatory cells inhibits liver failure and induces regeneration by acting at one or more of the stages of liver failure below.

During the priming phase, numerous injury signals are generated as a result of the underlying injury, these include activators of toll like receptors, complement degradation products, and Damage Associated Molecular Patterns (DAMPs). These signals stimulate various cells, primarily Kupffer cells, to produce cytokines and growth factors such as IL-6, TNF-alpha, and HGF, which induce entry of hepatocytes into cell cycle. The importance of these molecular signals in the initiation of liver regeneration is highlighted by knockout studies. Cressmann et al demonstrated in a partial hepatectomy IL-6 knockout model blockade of liver regeneration that was associated with blunted exit from G0 phase of cell cycle in hepatocytes of these mice but not in nonparenchymal liver cells. Furthermore, they conclusively showed the importance of IL-6 in that a single preoperative dose of recombinant IL-6 restored post-injury hepatocyte entry into G1/2 to levels observed in wild-type mice and restored biochemical function. Cressman, D. E., et al., Liver failure and defective hepatocyte regeneration in interleukin-6-deficient mice. Science, 1996, 274(5291): p. 1379-83. NF-kappa B is a major downstream effector of various inflammatory cytokines including TNF-alpha and IL-6. Malato et al. generated hepatic specific knockout mice in which the inhibitor of NF-kappa B, IKK2, was ablated, thus giving rise to a higher level of background NF-kappa B activation. In these mice partial hepatectomy resulted in accelerated entry of hepatocytes into cell cycle. Malato, Y., et al., Hepatocyte-specific inhibitor-of-kappaB-kinase deletion triggers the innate immune response and promotes earlier cell proliferation during liver regeneration. Hepatology, 2008, 47(6): p. 2036-50. The role of a variety of inflammatory or “danger” associated pathways in the initial priming of hepatocyte proliferation after injury has been confirmed using DNA microarray analysis of genes associated with these signaling pathways such as STAT, p38MAPK, and Ras/ERK. Li, M., et al., Study on the activity of the signaling pathways regulating hepatocytes from G0 phase into G1 phase during rat liver regeneration. Cell Mol Biol Lett, 2014, 19(2): p. 181-200. The disclosure teaches that depending on patient need various immunological interventions can be performed at this stage. For example, if the goal of the practitioner of the disclosure is to upregulate extent of hepatocyte regeneration, innate immune stimulators may be administered such as TLR agonists, or BCG, or beta glucan. It is to be noted that these should not be stimulators of robust inflammation that would be deleterious. In one embodiment, stimulators of TLRs may be added together with cells such as mesenchymal stem cells, which would suppress some aspects of the inflammatory response stimulated by TLR activators. In another embodiment monocytes may be added to the hepatic circulation or intrahepatically in order to augment extent of innate stimulation occurring. In other aspects, dendritic cells may be added.

The Proliferation Phase of hepatic regeneration is associated with “primed” hepatocytes leaving G1 stage of cell cycle and entering S phase, which is accompanied by phosphorylation of the retinoblastoma protein (pRb) and by up-regulated expression of a number of proliferation associated genes including cyclin E, cyclin A, and DNA polymerase. Fan, G., et al., Modulation of retinoblastoma and retinoblastoma-related proteins in regenerating rat liver and primary hepatocytes. Cell Growth Differ, 1995, 6(11): p. 1463-76; Spiewak Rinaudo, J. A. and S. S. Thorgeirsson, Detection of a tyrosine-phosphorylated form of cyclin A during liver regeneration. Cell Growth Differ, 1997, 8(3): p. 301-9. Key cytokines involved in stimulation of proliferation of the hepatocytes include hepatocyte growth factor (HGF) and epidermal growth factor (EGF). HGF is produced by mesenchymal cells, hepatic stellate cells, and liver sinusoidal endothelial cells as a pro-protein, which acts both systemically and locally. DeLeve, L. D., Liver sinusoidal endothelial cells and liver regeneration. J Clin Invest, 2013, 123(5): p. 1861-6; Maher, J. J., Cell-specific expression of hepatocyte growth factor in liver. Upregulation in sinusoidal endothelial cells after carbon tetrachloride. J Clin Invest, 1993, 91(5): p. 2244-52. Systemic elevations in HGF are observed after partial hepatectomy, Matsumoto, K., et al., Serial changes of serum growth factor levels and liver regeneration after partial hepatectomy in healthy humans. Int J Mol Sci, 2013, 14(10): p. 20877-89., whereas local HGF is released from its latent form, which is often bound to extracellular matrix proteins. Nakamura, T., et al., Hepatocyte growth factor twenty years on: Much more than a growth factor. J Gastroenterol Hepatol, 2011, 26 Suppl 1: p. 188-202. Activation of HGF occurs typically via enzymatic cleavage mediated by urokinase type plasminogen activator (uPA). Mars, W. M., et al., Immediate early detection of urokinase receptor after partial hepatectomy and its implications for initiation of liver regeneration. Hepatology, 1995, 21(6): p. 1695-701; Shanmukhappa, K., et al., Urokinase-type plasminogen activator supports liver repair independent of its cellular receptor. BMC Gastroenterol, 2006, 6: p. 40. The importance of HGF in the Proliferation Phase of liver regeneration is observed in animals where the HGF receptor c-MET is conditionally inactivated, which display a reduction in hepatocyte entry into the S phase of cell cycle post injury. Borowiak, M., et al., Met provides essential signals for liver regeneration. Proc Natl Acad Sci USA, 2004, 101(29): p. 10608-13. EGF signaling has also been demonstrated to be involved in entry into the proliferative phase post injury. Natarajan et al. performed perinatal deletion of EGFR in hepatocytes prior to partial hepatectomy. They showed that after hepatic injury mice lacking EGFR in the liver had an increased mortality accompanied by increased levels of serum transaminases indicating liver damage. Liver regeneration was delayed in the mutants because of reduced hepatocyte proliferation. Analysis of cell cycle progression in EGFR-deficient livers indicated a defective G(1)-S phase entry with delayed transcriptional activation and reduced protein expression of cyclin D1 followed by reduced cdk2 and cdk1 expression. Natarajan, A., B. Wagner, and M. Sibilia, The EGF receptor is required for efficient liver regeneration. Proc Natl Acad Sci USA, 2007, 104(43): p. 17081-6. Immunologically intervening in this stage would require the administration of immune cells producing growth factors. Such cells could be alternatively activated macrophages, or monocytes that have been pretreated with stimuli to increase production of growth factors such as those mentioned above including HGF. One method of stimulating immune cells to produce such growth factors includes culture with IGIV, or stimulation with hypoxia. It is further one embodiment of the disclosure to stimulate lymphocytes to produce growth factors by various in vitro culture techniques. For example, stimulation of allogeneic or autologous lymphocytes by culture with anti-CD3 and anti-CD28 in the presence of hepatocytes can be used to stimulate growth factor production that is beneficial for hepatocyte proliferation in vivo.

The termination phase of liver regeneration occurs when the normal liver-mass/body-weight ratio of 2.5% has been restored. Nygard, I. E., et al., The genetic regulation of the terminating phase of liver regeneration. Comp Hepatol, 2012, 11(1): p. 3. While in the Initiation Phase of liver regeneration, several inflammatory cytokines are critical, in the Termination Phase, antiinflammatory cytokines such as IL-10, Mosser, D. M. and X. Zhang, Interleukin-10: new perspectives on an old cytokine. Immunol Rev, 2008. 226: p. 205-18, are upregulated, which dampen proliferative stimuli. Yin, S., et al., Enhanced liver regeneration in IL-10-deficient mice after partial hepatectomy via stimulating inflammatory response and activating hepatocyte STAT3. Am J Pathol, 2011, 178(4): p. 1614-21. Additionally, cytokines with direct antiproliferative activity such as TGF-beta are generated, which result in cell cycle arrest of proliferating hepatocytes. In this phase immunological intervention may be to inhibit the arrest of hepatocyte proliferation, such as utilization of Th17 cells that inhibit TGF-beta production, or administration of cells that are fibrinolytic and express MMPs, such cells include lymphocytes, pretreated lymphocytes, and macrophages. Tregs produce latent TGF-β1 in association with a transmembrane protein called glycoprotein A repetitions predominant (GARP). Additionally, administration of MSC that are induced to express MMPs may be performed.

“Treat” or “treatment” means improving the symptoms and ameliorating autoimmune, septic, or pulmonary disease. Additionally, “treat” means improving ischemic conditions. Methods for measuring the rate of “treatment” efficacy are known in the art and include, for example, assessment of inflammatory cytokines.

“Angiogenesis” means any alteration of an existing vascular bed or the formation of new vasculature, which benefits tissue perfusion. This includes the formation of new vessels by sprouting of endothelial cells from existing blood vessels or the remodeling of existing vessels to alter size, maturity, direction or flow properties to improve blood perfusion of tissues. As used herein the terms, “angiogenesis,” “revascularization,” “increased collateral circulation,” and “regeneration of blood vessels” are considered as synonymous.

“Mesenchymal stem cell” or “MSC” refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. As used herein, “mesenchymal stromal cell” or “MSC” can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. As used herein, “mesenchymal stromal cell” or “MSC” includes cells that are CD34 positive upon initial isolation from tissue but are similar to cells described about phenotypically and functionally. As used herein, “MSC” includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. As used herein, “mesenchymal stromal cell” or “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, Stempeuce10A, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

While classical liver regeneration is mediated by hepatocytes in certain situations, such as in liver failure, the ability of the hepatocytes to mediate regeneration is limited and liver progenitor cells (LPCs) must carry out the process. Fausto, N., J. S. Campbell, and K. J. Riehle, Liver regeneration. Hepatology, 2006, 43(2 Suppl 1): p. S45-53; Miyaoka, Y. and A. Miyajima, To divide or not to divide: revisiting liver regeneration. Cell Div, 2013, 8(1): p. 8. The concept of a LPC, which took over regenerative function when hepatocyte multiplication is stunted, was first demonstrated in 1956 when Farber treated rats with various liver carcinogens that blocked division of hepatocytes. Farber, E., Similarities in the sequence of early histological changes induced in the liver of the rat by ethionine, 2-acetylamino-fluorene, and 3′-methyl-4-dimethylaminoazobenzene. Cancer Res, 1956, 16(2): p. 142-8. He discovered the existence of “Oval Cells” which were subsequently demonstrated to act as LPC having ability to differentiate into both hepatocytes and biliary cells. Evarts, R. P., et al., A precursor-product relationship exists between oval cells and hepatocytes in rat liver. Carcinogenesis, 1987, 8(11): p. 1737-40. LPC are found in the canals of Hering and bile ductules in human liver and found increased in patients with chronic liver disease. Libbrecht, L. and T. Roskams, Hepatic progenitor cells in human liver diseases. Semin Cell Dev Biol, 2002, 13(6): p. 389-96. It is unclear what the origin of LPCs is, whether they derive from local cells, or directly from MSCs, Banas, A., et al., Adipose tissue-derived mesenchymal stem cells as a source of human hepatocytes. Hepatology, 2007, 46(1): p. 219-28, particularly bone marrow derived MSCs, Petersen, B. E., et al., Bone marrow as a potential source of hepatic oval cells. Science, 1999. 284(5417): p. 1168-70, but the cellular mechanisms are poorly understood. Margini, C., et al., Bone marrow derived stem cells for the treatment of end-stage liver disease. World J Gastroenterol, 2014, 20(27): p. 9098-9105. In 2000 Theise et al. found hepatocytes and cholangiocytes derived from extrahepatic circulating stem cells in the livers of female patients who had undergone therapeutic bone marrow transplantations. In the two female recipients from male donors and four male recipients from female donors hepatocyte and cholangiocyte engraftment ranged from 4% to 43% and from 4% to 38%, respectively. Theise, N. D., et al., Liver from bone marrow in humans. Hepatology, 2000, 32(1): p. 11-6. Given the potent regenerative nature of the liver, combined with the possibility that extrahepatic cellular sources may contribute to regeneration, numerous attempts have been made to utilize cellular therapy for treatment of liver failure. The original hepatic cellular therapies involved the administration of allogeneic hepatocytes, which was attempted in animal models more than 30 years ago and is experimentally used clinically. Unfortunately, major hurdles exist that block this procedures from routine use, specifically: a) low number of suitable donors; b) extremely poor hepatocyte viability after transplantation, with some groups as low as 30%; and c) need for continuous immune suppression which possesses inherent adverse effects. Filippi, C. and A. Dhawan, Current status of human hepatocyte transplantation and its potential for Wilson's disease. Ann N Y Acad Sci, 2014, 1315: p. 50-5. In one embodiment, stimulation of LPC may be performed by administration of immune cells that provide growth factor support for these cells. This includes administration of cord blood mononuclear cells, or monocytes that have been cultured to possess augmented HGF and other hepatogenic growth factors. In some aspects, T regulatory cells are utilized together with LPC to facilitate engraftment, increase viability, and allow for enhanced regeneration.

It is known in the art that MSC are capable of possessing some activity against liver failure, however these have not been harnessed properly in the clinical setting. One of skill in the art is referred to the examples below of MSC use in liver failure, which the MSC can be manipulated immunologically as described herein to induce optimized therapeutic effects. Mesenchymal stem cells (MSCs) are adult stem cells with self-renewing abilities, Jackson, L., et al., Adult mesenchymal stem cells: differentiation potential and therapeutic applications. J Postgrad Med, 2007, 53(2): p. 121-7, and have been shown to differentiate into a wide range of tissues including mesoderm- and nonmesoderm-derived, id.; Pittenger, M. F., et al., Multilineage potential of adult human mesenchymal stem cells. Science, 1999, 284(5411): p. 143-7, such as hepatocytes. Banas, A., et al., Rapid hepatic fate specification of adipose-derived stem cells and their therapeutic potential for liver failure. J Gastroenterol Hepatol, 2009, 24(1): p. 70-7; Lee, K. D., et al., In vitro hepatic differentiation of human mesenchymal stem cells. Hepatology, 2004, 40(6): p. 1275-84; Cho, K. A., et al., Mesenchymal stem cells showed the highest potential for the regeneration of injured liver tissue compared with other subpopulations of the bone marrow. Cell Biol Int, 2009, 33(7): p. 772-7; Hong, S. H., et al., In vitro differentiation of human umbilical cord blood-derived mesenchymal stem cells into hepatocyte-like cells. Biochem Biophys Res Commun, 2005, 330(4): p. 1153-61; Ishikawa, T., et al., Stem cells for hepatic regeneration: the role of adipose tissue derived mesenchymal stem cells. Curr Stem Cell Res Ther, 2010, 5(2): p. 182-9; Seo, M. J., et al., Differentiation of human adipose stromal cells into hepatic lineage in vitro and in vivo. Biochem Biophys Res Commun, 2005, 328(1): p. 258-64. MSCs are capable of entering and maintaining satellite cell niches, particularly in hematopoiesis, Crisan, M., et al., A perivascular origin for mesenchymal stem cells in multiple human organs. Cell Stem Cell, 2008, 3(3): p. 301-13; Tavian, M. and B. Peault, Embryonic development of the human hematopoietic system. Int J Dev Biol, 2005, 49(2-3): p. 243-50, and are key in tissue repair and regeneration, aging, and regulating homeostasis. Peault, B., et al., Stem and progenitor cells in skeletal muscle development, maintenance, and therapy. Mol Ther, 2007, 15(5): p. 867-77; Aggarwal, S. and M. F. Pittenger, Human mesenchymal stem cells modulate allogeneic immune cell responses. Blood, 2005, 105(4): p. 1815-22; Caplan, A. I., Adult mesenchymal stem cells for tissue engineering versus regenerative medicine. J Cell Physiol, 2007, 213(2): p. 341-7; Chamberlain, G., et al., Concise review: mesenchymal stem cells: their phenotype, differentiation capacity, immunological features, and potential for homing. Stem Cells, 2007, 25(11): p. 2739-49. In the case of liver failure, MSCs can aid in regeneration of hepatic tissue, Banas, A., et al., IFATS collection: in vivo therapeutic potential of human adipose tissue mesenchymal stem cells after transplantation into mice with liver injury. Stem Cells, 2008, 26(10): p. 2705-12; Kharaziha, P., et al., Improvement of liver function in liver cirrhosis patients after autologous mesenchymal stem cell injection: a phase I-II clinical trial. Eur J Gastroenterol Hepatol, 2009, 21(10): p. 1199-205; Kuo, T. K., et al., Stem cell therapy for liver disease: parameters governing the success of using bone marrow mesenchymal stem cells. Gastroenterology, 2008, 134(7): p. 2111-21, 2121 el-3; Chang, Y. J., et al., Mesenchymal stem cells facilitate recovery from chemically induced liver damage and decrease liver fibrosis. Life Sci, 2009, 85(13-14): p. 517-25; Lu, L. L., et al., Isolation and characterization of human umbilical cord mesenchymal stem cells with hematopoiesis-supportive function and other potentials. Haematologica, 2006, 91(8): p. 1017-26; Mohamadnejad, M., et al., Phase 1 trial of autologous bone marrow mesenchymal stem cell transplantation in patients with decompensated liver cirrhosis. Arch Iran Med, 2007, 10(4): p. 459-66; Terai, S., et al., Improved liver function in patients with liver cirrhosis after autologous bone marrow cell infusion therapy. Stem Cells, 2006, 24(10): p. 2292-8, and their interactions with the immune system, Chang, C. J., et al., Placenta-derived multipotent cells exhibit immunosuppressive properties that are enhanced in the presence of interferon-gamma. Stem Cells, 2006, 24(11): p. 2466-77; Iyer, S. S. and M. Rojas, Anti-inflammatory effects of mesenchymal stem cells: novel concept for future therapies. Expert Opin Biol Ther, 2008, 8(5): p. 569-81; Nauta, A. J. and W. E. Fibbe, Immunomodulatory properties of mesenchymal stromal cells. Blood, 2007, 110(10): p. 3499-506; Uccelli, A., V. Pistoia, and L. Moretta, Mesenchymal stem cells: a new strategy for immunosuppression? Trends Immunol, 2007, 28(5): p. 219-26; Wolbank, S., et al., Dose-dependent immunomodulatory effect of human stem cells from amniotic membrane: a comparison with human mesenchymal stem cells from adipose tissue. Tissue Eng, 2007, 13(6): p. 1173-83; Wolf, D. and A. M. Wolf, Mesenchymal stem cells as cellular immunosuppressants. Lancet, 2008, 371(9624): p. 1553-4; Shi, M., Z. W. Liu, and F. S. Wang, Immunomodulatory properties and therapeutic application of mesenchymal stem cells. Clin Exp Immunol, 2011, 164(1): p. 1-8, have potential as adjuvants during organ transplants, Sordi, V. and L. Piemonti, Therapeutic plasticity of stem cells and allograft tolerance. Cytotherapy, 2011, 13(6): p. 647-60, including liver transplantation. Popp, F. C., et al., Mesenchymal stem cells as immunomodulators after liver transplantation. Liver Transpl, 2009, 15(10): p. 1192-8.

In one embodiment, T regulatory cells are utilized to enhance activity of MSC in treatment of liver failure.

Currently there several MSC-based therapies that have received governmental approvals including Prochymal™ which was registered in Canada and New Zealand for treatment of graft versus host disease. Kurtzberg, J., et al., Allogeneic human mesenchymal stem cell therapy (remestemcel-L, Prochymal) as a rescue agent for severe refractory acute graft-versus-host disease in pediatric patients. Biol Blood Marrow Transplant, 2014, 20(2): p. 229-35; Kellathur, S. N. and H. X. Lou, Cell and tissue therapy regulation: worldwide status and harmonization. Biologicals, 2012, 40(3): p. 222-4. Although in terms of clinical translation bone marrow MSC are the most advanced, several other sources of MSC are known which possess various properties that may be useful for specific conditions. Bone marrow is also a source for hematopoietic stem cells (HSCs), which have also been used for liver regeneration. Likewise, human placenta is an easily accessible source of abundant MSCs, which can be differentiated in vitro. Finally, MSCs with tissue regenerative abilities can also be isolated from adipose tissue and induced to hepatocytes in large numbers.

Early studies suggested that out of the hepatic regenerative cells found in the bone marrow that the MSC component is the most regenerative cell type as compared to other cell types such as hematopoietic stem cells. Cho, K. A., et al., Mesenchymal stem cells showed the highest potential for the regeneration of injured liver tissue compared with other subpopulations of the bone marrow. Cell Biol Int, 2009, 33(7): p. 772-7. Given the fact that BM-MSC are capable of differentiating into various tissues in vitro, combined with the putative bone marrow origin of the hepatic-repairing oval cell, Petersen, B. E., et al., Bone marrow as a potential source of hepatic oval cells. Science, 1999, 284(5417): p. 1168-70, investigators sought to determine whether BM-MSC could be induced to differentiate into hepatocyte cells in vitro through culture in conditions that would imitate hepatic regeneration. Lee et al developed a 2-step protocol for hepatocyte differentiation using culture in hepatocyte growth factor, followed by oncostatin M. After 4 weeks of induction the investigators reported the spindle-like BM-MSC taking a cuboidal morphology, which is characteristic of hepatocytes. Furthermore the differentiating cells were seen to initiate expression of hepatic-specific genes in a time-dependent manner correlating with morphological changes. From a functional perspective, the generated hepatocytes exhibited features of liver cells, specifically albumin production, glycogen storage, urea secretion, uptake of low-density lipoprotein, and phenobarbital-inducible cytochrome P450 activity. Lee, K. D., et al., In vitro hepatic differentiation of human mesenchymal stem cells. Hepatology, 2004, 40(6): p. 1275-84. To improve yield and potency of BM-MSC generated hepatocytes, Chen et al utilized conditioned media from cultured hepatocytes as part of the differentiation culture conditions. They reported that BM-MSC cultures in the differentiation conditions started taking an epithelioid, binucleated morphology at days 10 and 20. Gene assessment revealed increase in AFP, HNF-3beta, CK19, CK18, ALB, TAT, and G-6-Pase mRNA, which was confirmed at the protein levels. Additionally, the cells started taking a functional phenotype similar to hepatocytes, including, hepatocyte-like cells by culture in conditioned medium further demonstrated in vitro functions characteristic of liver cells, including glycogen storage, and urea secretion activities. In vivo relevance of these artificially generated hepatic like cells was seen in that restoration of albumin activity and suppression of liver enzymes was seen upon transplantation to immune deficient animals exposed to chemically induced liver injury. Chen, Y., et al., In vitro differentiation of mouse bone marrow stromal stem cells into hepatocytes induced by conditioned culture medium of hepatocytes. J Cell Biochem, 2007, 102(1): p. 52-63. In accordance with the concept that injured tissue mediates MSC activation and subsequent repair, Mohsin et al demonstrated that coculture of BM-MSC with chemically-injured hepatocytes augments hepatic differentiation as compared to coculture with naïve hepatocytes. Mohsin, S., et al., Enhanced hepatic differentiation of mesenchymal stem cells after pretreatment with injured liver tissue. Differentiation, 2011, 81(1): p. 42-8.

For guidance as to the clinical dosage of T regulatory cells, the disclosure teaches that doses similar to, or higher than those used for MSC in treatment of liver failure may be utilized. Example clinical trials are discussed below. Clinical trials utilizing BM-MSC have shown an excellent safety profile, with various levels of efficacy in liver failure. Mohamadnejad et al., Mohamadnejad, M., et al., Phase 1 trial of autologous bone marrow mesenchymal stem cell transplantation in patients with decompensated liver cirrhosis. Arch Iran Med, 2007, 10(4): p. 459-66, conducted a 4 patient study with decompensated liver cirrhosis. Patient bone marrow was aspirated, mesenchymal stem cells were cultured, and a mean 31.73×10(6) mesenchymal stem cells were infused through a peripheral vein. There were no side effects in the patients during follow-up. The model for end-stage liver disease (MELD) scores of patients 1, and 4 improved by four and three points, respectively by the end of follow-up. Furthermore, the quality of life of all four patients improved by the end of follow-up. Using SF-36 questionnaire, the mean physical component scale increased from 31.44 to 65.19, and the mean mental component scale increased from 36.32 to 65.55. Another study treated eight patients (four hepatitis B, one hepatitis C, one alcoholic, and two cryptogenic) with end-stage liver disease having MELD score > or =10 were included. Autologous BM-MSCs were taken from iliac crest. Approximately, 30-50 million BM-MSCs were proliferated and injected into peripheral or the portal vein. Subsequent to experiment the MELD Score was decreased from 17.9+/−5.6 to 10.7+/−6.3 (P<0.05) and prothrombin complex from international normalized ratio 1.9+/−0.4 to 1.4+/−0.5 (P<0.05). Serum creatinine decreased from 114+/−35 to 80+/−18 micromol/l (P<0.05). This trial supports the safety with signal of efficacy of the BM-MSC activity in liver failure clinically.

A larger trial of autologous BM-MSC focused on patients with liver failure associated with hepatitis B infection. Peng, L., et al., Autologous bone marrow mesenchymal stem cell transplantation in liver failure patients caused by hepatitis B: short-term and long-term outcomes. Hepatology, 2011, 54(3): p. 820-8. Part of the rational was previous studies showing that BM-MSC derived hepatocytes are resistant to hepatitis B infection. Xie, C., et al., Human bone marrow mesenchymal stem cells are resistant to HBV infection during differentiation into hepatocytes in vivo and in vitro. Cell Biol Int, 2009, 33(4): p. 493-500. Peng et al., supra, treated 53 patients and as controls used 105 patients matched for age, sex, and biochemical indexes, including alanine aminotransferase (ALT), albumin, total bilirubin (TBIL), prothrombin time (PT), and MELD score. In the 2-3 week period after cell administration, efficacy was observed based on levels of ALB, TBIL, and PT and MELD score, compared with those in the control group. Safety of the procedure was demonstrated in that there were no differences in incidence of hepatocellular carcinoma (HCC) or mortality between the treated and control groups at 192 weeks. Unfortunately, liver function between the two groups was also similar at 192 weeks, suggesting the beneficial effects of BM-MSC were transient in nature. Supporting the possibility of transient effects of BM-MSC was a 27 patient study in patients with decompensated cirrhosis in which 15 patients received BM-MSC and 12 patients received placebo. The absolute changes in Child scores, MELD scores, serum albumin, INR, serum transaminases and liver volumes did not differ significantly between the MSC and placebo groups at 12 months of follow-up. Unfortunately, the publication did not provide 3 or 6 month values. Mohamadnejad, M., et al., Randomized placebo-controlled trial of mesenchymal stem cell transplantation in decompensated cirrhosis. Liver Int, 2013, 33(10): p. 1490-6. In contrast, a more recent study administered BM-MSC into 12 patients (11 males, 1 female) with baseline biopsy-proven alcoholic cirrhosis who had been alcohol free for at least 6 months. Jang, Y. O., et al., Histological improvement following administration of autologous bone marrow-derived mesenchymal stem cells for alcoholic cirrhosis: a pilot study. Liver Int, 2014, 34(1): p. 33-41. A 3-month assessment histological improvement and reduction of fibrosis was quantified according to the Laennec fibrosis scoring scale in 6 of 11 patients. Additionally, at 3 months post cell administration, the Child-Pugh score improved significantly in ten patients and the levels of transforming growth factor-β1, type 1 collagen and a-smooth muscle actin significantly decreased (as assessed by real-time reverse transcriptase polymerase chain reaction) after BM-MSCs therapy. Overall, the different underlying conditions, route of administration, and time points of assessments between studies makes it difficult to draw solid conclusions, although it appears that some therapeutic effect exists, although longevity of effect is not known.

Given that one possibility for the lack of efficacy long term in the previous study may be inappropriate level of hepatocyte differentiation in vivo, Amer et al. conducted a clinical trial where BM-MSC were pre-differentiated toward the hepatocyte lineage by a culture cocktail containing HGF. Amer, M. E., et al., Clinical and laboratory evaluation of patients with end-stage liver cell failure injected with bone marrow-derived hepatocyte-like cells. Eur J Gastroenterol Hepatol, 2011, 23(10): p. 936-41. They conducted a 40 patient trial in hepatitis C patients in which 20 patients were treated with partially differentiated cells either intrasplenically or intrahepatically and 20 patients received placebo control. At the 3 and 6 month time points a significant improvement in ascites, lower limb edema, and serum albumin, over the control group was observed. Additionally significant benefit was quantified in the Child-Pugh and MELD scores. No difference was observed between intrahepatic or intrasplenic administration. This study demonstrates the potential of semi-differentiated hepatocytes from BM-MSC to yield therapeutic benefit without reported adverse effects. In one embodiment, T regulatory cells are administered together with MSC that have been differentiated towards the hepatic lineage

One of the first clinical uses of BMMC in the liver involved purification of CD133 positive cells prior to administration, with the notion that CD133 selects for cells with enhanced regenerative potential. Handgretinger, R. and S. Kuci, CD133-Positive Hematopoietic Stem Cells: From Biology to Medicine. Adv Exp Med Biol, 2013, 777: p. 99-111. Additionally, the CD133 subset of bone marrow cells may represent a hepatogenic precursor cell since cells of this phenotype are mobilized from the bone marrow subsequent to partial hepatectomy. Zocco, M. A., et al., CD133+ stem cell mobilization after partial hepatectomy depends on resection extent and underlying disease. Dig Liver Dis, 2011, 43(2): p. 147-54; Harb, R., et al., Bone marrow progenitor cells repair rat hepatic sinusoidal endothelial cells after liver injury. Gastroenterology, 2009, 137(2): p. 704-12; Gehling, U. M., et al., Partial hepatectomy induces mobilization of a unique population of haematopoietic progenitor cells in human healthy liver donors. J Hepatol, 2005, 43(5): p. 845-53. Another interesting point is that CD133 has been reported by some to be expressed on oval cells in the liver, although the bone marrow origin is controversial. Rountree, C. B., et al., A CD133-expressing murine liver oval cell population with bilineage potential. Stem Cells, 2007, 25(10): p. 2419-29; Rountree, C. B., et al., Isolation of CD133+ liver stem cells for clonal expansion. J Vis Exp, 2011(56); Yovchev, M. I., et al., Novel hepatic progenitor cell surface markers in the adult rat liver. Hepatology, 2007, 45(1): p. 139-49. In 2005 am Esch et al described 3 patients subjected to intraportal administration of autologous CD133(+) BMSCs subsequent to portal venous embolization of right liver segments, used to expand left lateral hepatic segments. Computerized tomography scan volumetry revealed 2.5-fold increased mean proliferation rates of left lateral segments compared with a group of three consecutive patients treated without application of BMSCs. am Esch, J. S., 2nd, et al., Portal application of autologous CD133+ bone marrow cells to the liver: a novel concept to support hepatic regeneration. Stem Cells, 2005. 23(4): p. 463-70. In 2012 the same group reported on 11 patients treated with this procedure and 11 controls. They reported that mean hepatic growth of segments II/III 14 days after portal vein embolization in the group that received CD133 cells was significantly higher (138.66 mL±66.29) when compared with the control group (62.95 mL±40.03; P=0.004). Post hoc analysis revealed a better survival for the group that received cells as compared to the control. A similar study by another group involved 6 patients receiving CD133 cells to accelerate left lateral segment regeneration, with 7 matched control patients. The increase of the mean absolute future liver remnant volume (FLRV) in the treated group from 239.3 mL +/−103.5 to 417.1 mL +/−150.4 was significantly higher than that in the control group, which was from 286.3 mL +/−77.1 to 395.9 mL +/−94.1. The daily hepatic growth rate in the treated group (9.5 mL/d +/−4.3) was significantly higher to that in the control group (4.1 mL/d +/−1.9) (P=0.03). Furthermore, time to surgery was 27 days +/−11 in the treated group and 45 days +/−21 in the control group (P=0.057). These data suggest that in the clinical situation, CD133 cells isolated from BMMC appear to accelerate liver regeneration. In one embodiment, liver failure is treated by combination of autologous bone marrow mononuclear cells, or purified subsets (eg CD34, CD133, or aldehyde dehydrogenase high) together with allogeneic T regulatory cells. In some embodiments, T regulatory cells may be autologous.

Another purified cell type from BMMC is CD34 expressing cells, which conventionally are known to possess the hematopoietic stem cell compartment. Sidney, L. E., et al., Concise review: evidence for CD34 as a common marker for diverse progenitors. Stem Cells, 2014, 32(6): p. 1380-9. Additionally, similar to CD133, CD34 is found on oval cells in the liver, suggesting possibility that bone marrow derived CD34 cells play a role in liver regeneration when hepatocyte proliferation is inhibited. Crosby, H. A., D. A. Kelly, and A. J. Strain, Human hepatic stem-like cells isolated using c-kit or CD34 can differentiate into biliary epithelium. Gastroenterology, 2001, 120(2): p.534-44; Theise, N. D., et al., Derivation of hepatocytes from bone marrow cells in mice after radiation-induced myeloablation. Hepatology, 2000, 31(1): p. 235-40. Gordon et al., reported 5 patients with liver failure that were treated with isolated CD34 positive cells. Gordon, M. Y., et al., Characterization and clinical application of human CD34+ stem/progenitor cell populations mobilized into the blood by granulocyte colony-stimulating factor. Stem Cells, 2006, 24(7): p. 1822-30. Interestingly, instead of collected the cells from bone marrow harvest, the investigators mobilized the bone marrow cells by treatment with G-CSF. The investigators first demonstrated that these CD34 cells were capable of differentiating in vitro into albumin producing hepatocyte-like cells. A pilot clinical investigation was attempted in 5 patients with liver failure. The CD34 cells were injected into the portal vein (three patients) or hepatic artery (two patients). No complications or specific side effects related to the procedure were observed. Three of the five patients showed improvement in serum bilirubin and four of five in serum albumin. A subsequent publication by the same group reported the improvement in bilirubin levels was maintained for 18 months. Levicar, N., et al., Long-term clinical results of autologous infusion of mobilized adult bone marrow derived CD34+ cells in patients with chronic liver disease. Cell Prolif, 2008, 41 Suppl 1: p. 115-25. A subsequent case report by Gasbarrini et al. described use of autologous CD34+ BMIVIC administered via the portal vein as a rescue treatment in an alcoholic patient with nimesulide -induced acute liver failure. Id. A liver biopsy performed at 20 days following infusion showed augmentation of hepatocyte replication around necrotic foci; there was also improvement in synthetic liver function within the first 30 days.

Subsequent to the initial studies on CD133 and CD34 cells, investigators assessed the effects of unpurified BMMC on liver failure. Terai et al., treated 9 patients with liver cirrhosis from a variety of causes with autologous BMIVIC administered intravenously. Terai, S., et al., Improved liver function in patients with liver cirrhosis after autologous bone marrow cell infusion therapy. Stem Cells, 2006, 24(10): p. 2292-8. Significant improvements in serum albumin levels and total protein were observed at 24 weeks after BMMC therapy. Significantly improved Child-Pugh scores were seen at 4 and 24 weeks. alpha-Fetoprotein and proliferating cell nuclear antigen (PCNA) expression in liver biopsy tissue was significantly elevated after BMIVIC infusion. No major adverse effects were noted. A subsequent study in alcohol associated decompensated liver failure examined effects of autologous BMIVIC administered intraportally in 28 patients compared to 30 patients receiving standard medical care. After 3 months, 2 and 4 patients died in the BMMC and control groups, respectively. Adverse events were equally distributed between groups. The MELD score improved in parallel in both groups during follow-up. Comparing liver biopsy at 4 weeks to baseline, steatosis improved, and proliferating HPC tended to decrease in both groups. Spahr, L., et al., Autologous bone marrow mononuclear cell transplantation in patients with decompensated alcoholic liver disease: a randomized controlled trial. PLoS One, 2013, 8(1): p. e53719. It is unclear why this larger study generated a negative outcome compared to the initial smaller study. Interestingly in another study in which 32 patients with decompensating liver cirrhosis were treated with autologous BMMC and 15 patients received standard of care, significant improvements were observed. Specifically, improvements in ALT, AST, albumin, bilirubin and histological score where observed. The efficacy of BMMC transplantation lasted 3-12 months as compared with the control group. Serious complications such as hepatic encephalopathy and spontaneous bacterial peritonitis were also significantly reduced in BM-MNCs transfused patients compared with the controls. However, these improvements disappeared in 24 months after transplantation. Bai, Y. Q., et al., Outcomes of autologous bone marrow mononuclear cell transplantation in decompensated liver cirrhosis. World J Gastroenterol, 2014, 20(26): p. 8660-6. It is possible that effects of BMMC are transient in liver failure, lasting less than 12 months. For example, Lyra et al, reported on 10 patients with Child-Pugh B and C liver failure who received autologous BMMC. Bilirubin levels were lower at 1 (2.19+/−0.9) and 4 months (2.10+/−1.0) after cell transplantation that baseline levels (2.78+/−1.2). Lyra, A. C., et al., Feasibility and safety of autologous bone marrow mononuclear cell transplantation in patients with advanced chronic liver disease. World J Gastroenterol, 2007, 13(7): p. 1067-73. Albumin levels 4 months after BMIVIC infusion (3.73+/−0.5) were higher than baseline levels (3.47+/−0.5). International normalized ratio (INR) decreased from 1.48 (SD=0.23) to 1.43 (SD=0.23) one month after cell transplantation. A larger study by the same group utilizing similar methodology reported similar transient benefit. Lyra, A. C., et al., Infusion of autologous bone marrow mononuclear cells through hepatic artery results in a short-term improvement of liver function in patients with chronic liver disease: a pilot randomized controlled study. Eur J Gastroenterol Hepatol, 2010, 22(1): p. 33-42. Specifically, a 30 patient study was conducted with hepatic cirrhosis patients on the transplant list who were randomized to receive BMMC or supportive care. Child-Pugh score improved in the first 90 days in the cell therapy group compared with controls. The MELD score remained stable in the treated group but increased during follow-up in the control group. Albumin levels improved in the treatment arm, whereas they remained stable among controls in the first 90 days. Bilirubin levels increased among controls, whereas they decreased in the therapy arm during the first 60 days; INR RC differences between groups reached up to 10%. The changes observed did not persist beyond 90 days.

Other means of utilizing bone marrow stem cells for hepatic regeneration include stimulating mobilization of endogenous stem cells by providing agents such as G-CSF. Experimental studies to investigate the mobilization of HSCs for hepatocyte formation have yielded conflicting results, Cantz, T., et al., Reevaluation of bone marrow-derived cells as a source for hepatocyte regeneration. Cell Transplant, 2004, 13(6): p. 659-66; Jang, Y. Y., et al., Hematopoietic stem cells convert into liver cells within days without fusion. Nat Cell Biol, 2004, 6(6): p. 532-9; Kanazawa, Y. and I. M. Verma, Little evidence of bone marrow-derived hepatocytes in the replacement of injured liver. Proc Natl Acad Sci USA, 2003, 100 Suppl 1: p. 11850-3, but shitzu et al in 2012 showed beneficial effects in a murine model of acute liver failure. Shizhu, J., et al., Bone marrow mononuclear cell transplant therapy in mice with CCl4-induced acute liver failure. Turk J Gastroenterol, 2012, 23(4): p. 344-52. In one embodiment, administration of T regulatory cells together with bone marrow mobilization is performed for treatment of liver failure.

In one embodiment, cryopreserved cord blood bags (1 unit bags) are thawed and washed in CliniMACS buffer (Miltenyi Biotec, Bergish Gladbach, Germany) containing 0.5% HSA (Baxter Healthcare, Westlake Village, Calif.) in order to purify mononuclear cells. Subsequently, cells CD25+ cell enrichment is performed by positive selection using magnetic activated cell sorting (MACS) according to manufacturer's instructions (Miltenyi Biotec, Bergish Gladbach, Germany). Cells are check for viability and subsequently stimulated by co-cultured with CD3/28 co-expressing Dynabeads® (ClinExVivo™ CD3/CD28, Invitrogen Dynal A S, Oslo, Norway) at a 1 cell: 3 bead ratio9 and re-suspended at 1×106 cells/ml in X-VIVO 15 medium (Cambrex BioScience, Walkersville, Md.) supplemented with 10% human AB serum (Gemini Bio-Products, Sacramento, Calif.), 2 mM L-glutamine (Sigma, St. Louis, Mo.), 1% Penicillin-Streptomycin (Gibco/Invitrogen, Grand Island, N.Y.)] and 200 IU/ml interleukin (IL)-2 (CHIRON Corporation, Emeryville, Calif.). Ex vivo co-culture of the CD25+ cells and beads is performed in tissue culture flasks at 37° C. in a 5% CO2-in-air atmosphere. The CB-derived CD25+ enriched T-cells are maintained at 1×106 cells/ml by the addition of fresh medium and IL-2 (maintaining 200 IU/ml) every 48-72 hours. Addition of Wharton Jelly MSC was performed in some cultures. Said MSC where pre-plated at 50% confluency prior to addition of cord blood cells as described above.

Cell expansion for cells originating from any of the abovementioned tissues above takes place in clean room facilities purpose built for cell therapy manufacture and meeting GMP clean room classification. In a sterile class II biologic safety cabinet located in a class 10,000 clean production suite, cells were thawed under controlled conditions and washed in a 15 mL conical tube with 10 ML of complete DMEM-low glucose media (cDMEM) (GibcoBRL, Grand Island, N.Y.) supplemented with 20% Fetal Bovine Serum (Atlas) from dairy cattle confirmed to have no BSE % Fetal Bovine Serum specified to have Endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used is sequestered and one lot was used for all experiments. Cells are subsequently placed in a T-225 flask containing 45 mL of cDMEM and cultured for 24 hours at 37 .degree. C. at 5% CO2 in a fully humidified atmosphere. This allowed the MSC to adhere. Non-adherent cells were washed off using cDMEM by gentle rinsing of the flask. This resulted in approximately 6 million cells per initiating T-225 flask. The cells of the first flask were then split into 4 flasks. Cells were grown for 4 days after which approximately 6 million cells per flask were present (24 million cells total). This scheme was repeated but cells were not expanded beyond 10 passages, and were then banked in 6 million cell aliquots in sealed vials for delivery. All processes in the generation, expansion, and product production were performed under conditions and testing that was compliant with current Good Manufacturing Processes and appropriate controls, as well as Guidances issued by the FDA in 1998 Guidance for Industry: Guidance for Human Somatic Cell Therapy and Gene Therapy; the 2008 Guidance for FDA Reviewers and Sponsors Content and Review of Chemistry, Manufacturing, and Control (CMC) Information for Human Somatic Cell Therapy Investigational New Drug Applications (INDs); and the 1993 FDA points-to-consider document for master cell banks were all followed for the generation of the cell products described. Donor cells are collected in sterile conditions, shipped to a contract manufacturing facility, assessed for lack of contamination and expanded. The expanded cells are stored in cryovials of approximately 6 million cells/vial, with approximately 100 vials per donor. At each step of the expansion quality control procedures were in place to ensure lack of contamination or abnormal cell growth.

Without departing from the spirit of the disclosure, mesenchymal stem cells, as well as the culture of mesenchymal stem cells and T regulatory cells may be optimized to possess heightened immune modulatory properties. In one embodiment this may be performed by exposure of mesenchymal stem cells to hypoxic conditions, specifically hypoxic conditions can comprise an oxygen level of lower than 10%. In some embodiments, hypoxic conditions comprise up to about 7% oxygen. For example, hypoxic conditions can comprise up to about 7%, up to about 6%, up to about 5%, up to about 4%, up to about 3%, up to about 2%, or up to about 1% oxygen. As another example, hypoxic conditions can comprise up to 7%, up to 6%, up to 5%, up to 4%, up to 3%, up to 2%, or up to 1% oxygen. In some embodiments, hypoxic conditions comprise about 1% oxygen up to about 7% oxygen. For example, hypoxic conditions can comprise about 1% oxygen up to about 7% oxygen; about 2% oxygen up to about 7% oxygen; about 3% oxygen up to about 7% oxygen; about 4% oxygen up to about 7% oxygen; about 5% oxygen up to about 7% oxygen; or about 6% oxygen up to about 7% oxygen. As another example, hypoxic conditions can comprise 1% oxygen up to 7% oxygen; 2% oxygen up to 7% oxygen; 3% oxygen up to 7% oxygen; 4% oxygen up to 7% oxygen; 5% oxygen up to 7% oxygen; or 6% oxygen up to 7% oxygen. As another example, hypoxic conditions can comprise about 1% oxygen up to about 7% oxygen; about 1% oxygen up to about 6% oxygen; about 1% oxygen up to about 5% oxygen; about 1% oxygen up to about 4% oxygen; about 1% oxygen up to about 3% oxygen; or about 1% oxygen up to about 2% oxygen. As another example, hypoxic conditions can comprise 1% oxygen up to 7% oxygen; 1% oxygen up to 6% oxygen; 1% oxygen up to 5% oxygen; 1% oxygen up to 4% oxygen; 1% oxygen up to 3% oxygen; or 1% oxygen up to 2% oxygen. As another example, hypoxic conditions can comprise about 1% oxygen up to about 7% oxygen; about 2% oxygen up to about 6% oxygen; or about 3% oxygen up to about 5% oxygen. As another example, hypoxic conditions can comprise 1% oxygen up to 7% oxygen; 2% oxygen up to 6% oxygen; or 3% oxygen up to 5% oxygen. In some embodiments, hypoxic conditions can comprise no more than about 2% oxygen. For example, hypoxic conditions can comprise no more than 2% oxygen.

### Methods of Treating Liver Disorders

Some embodiments relate to a method of treating a liver disorder. As used herein, a liver disorder or a liver disease refers to liver cell injuries or damages caused by certain factors, which then potentially lead to liver dysfunction. According to the present invention, the compositions described herein can be used to ameliorate a liver disease or disorder. The term “liver damage(s)” used herein refers to liver with histological or biochemical dysfunction, as compared with normal liver. In a specific embodiment, as used herein, the term “liver damages” refers to liver lesions caused by alcoholic or non-alcoholic factors, such as high fat diet or obesity. In a specific embodiment, the term “liver damages” could be liver tissue damages with one or more characteristics selected from steatosis, lobular inflammation, hepatocyte ballooning, and vesicular fat droplets produced by liver cells. In a specific embodiment, the term “liver damages” can be biochemical dysfunction of liver, which can be determined from the activity of alanine aminotransferase (ALT) or aspartate transaminase (AST). Higher activity levels of ALT or AST indicate severer dysfunction of liver's biochemical functions.

A liver disorder may refer to any disease or disorder that affects the liver. Diseases or disorders of the liver are numerous, and more than one hundred types of liver disease have been characterized. Examples of liver disease include, but are not limited to, Alagille Syndrome; Alcohol-Related Liver Disease; Alpha-1 Antitrypsin Deficiency; Autoimmune Hepatitis; Benign Liver Tumors; Biliary Atresia; Cirrhosis; Galactosemia; Gilbert Syndrome; Hemochromatosis; Hepatitis A; Hepatitis B; Hepatitis C; Hepatocellular Carcinoma; Hepatic Encephalopathy; Liver Cysts; Liver Cancer; Newborn Jaundice; Non-Alcoholic Fatty Liver Disease (including nonalcoholic fatty liver and nonalcoholic steatohepatitis); Primary Biliary Cirrhosis (PBC); Primary Sclerosing Cholangitis (PSC); Reye Syndrome; Type I Glycogen Storage Disease and Wilson Disease.

In some embodiments, the method includes selecting a subject having or suspected of having a liver disorder. In some embodiments, a subject is selected who has a propensity for development of a liver disorder, for example, based on genetic or environmental propensities for developing a liver disorder. In some embodiments, treatment of a subject includes reducing symptoms of liver disorder, preventing symptoms of liver disorder, regenerating liver cells, or decreasing the likelihood of developing a liver disorder. As used herein, the terms “treating,” “treatment,” “therapeutic,” or “therapy” do not necessarily mean total cure or abolition of the disease or condition.

As used herein, the term “inhibit” refers to the reduction or prevention of the liver disorder. The reduction can be by 10%, 20%, 30%, 40%, 50%, 60%, 70%, 80%, 90%, or 100%, or an amount that is within a range defined by any two of the aforementioned values. As used herein, the term “delay” refers to a slowing, postponement, or deferment of an event, such as a liver disorder, to a time which is later than would otherwise be expected. The delay can be a delay of 10%, 20%, 30%, 40%, 50%, 60%, 70%, 80%, 90%, 100%, or an amount within a range defined by any two of the aforementioned values. The terms inhibit and delay are not to be construed as necessarily indicating a 100% inhibition or delay. A partial inhibition or delay may be realized.

In some embodiments, the method includes administering a pharmaceutical composition as described herein, wherein the pharmaceutical composition includes a population of activated T regulatory cells. In some embodiments, a population of activated T regulatory cells possesses enhanced production of liver regenerating factors by culture with allogeneic mesenchymal stem cells. In some embodiments, the population of T regulatory cells is or has been rendered activated to induce liver proliferation by enhanced ability to produce hepatocyte growth factor, and to enhance hepatic oval cell proliferation. In some embodiments, treating liver failure includes reducing liver fibrosis. As used herein, liver fibrosis refers to reduction in functional hepatocytes. In some embodiments, treating liver failure comprises stimulating proliferation of liver tissue growth after injury. As used herein, liver regeneration is defined as proliferation of hepatocytes.

As used herein, hepatic oval cell or liver progenitor cell is a cell responsible for generation of new hepatic tissue under conditions where hepatocytes stop proliferating in response to injury or damage.

In some embodiments, the T regulatory cells are derived from peripheral blood mononuclear cells, mobilized peripheral blood mononuclear cells, cord blood, menstrual blood, or adipose stromal vascular fraction cells. In some embodiments, the mobilized peripheral blood mononuclear cells are obtained from administration of G-CSF, flt-3 ligand, thrombopoietin, or Mozobil. In some embodiments, the activated T regulatory cells inhibit proliferation of naive T cells stimulated with a signal that activates proliferation. In some embodiments, the signal that activates proliferation is anti-CD3 and anti-CD28 beads, concanavalin A, PHA, or stimulation with alloreactive antigen presentation cells. In some embodiments, the activated T regulatory cells suppress maturation of dendritic cells. As used herein, maturation of dendritic cells is upregulation CD80, CD40, CD86, or HLA II. In some embodiments, the activated T regulatory cells express neuropilin-1, CTLA-4, CD25, CD39, CD73, CD105, CD127, FoxP3, GARP, GITR ligand, IL-10, or membrane bound TGF-beta. In some embodiments, the activated T regulatory cells are activated by exposure to vasoactive intestinal peptide, IL-10, TGF-beta, mesenchymal stem cell conditioned media, mesenchymal stem cell derived exosomes, BDNF, human chorionic gonadotropin, VEGF, CD3 or CD28 antibodies, hypoxic conditions, rapamycin, or angiopoietin. In some embodiments, the activated T regulatory cells are anergic T cells.

In some embodiments, the T regulatory cells are cocultured with type 2 monocytes, CD5 positive B cells, type 2 NKT cells, tolerogenic dendritic cells, gamma delta T cells, T cells with immune regulatory properties, CD34 cells, very small embryonic like stem cells, or Sertoli cells.

In some embodiments, the mesenchymal stem cells are in a mitotically inactivated state. In some embodiments, the T regulatory cells are treated with an immune modulator prior to administration. In some embodiments, the mesenchymal stem cells are cultured with an immune modulator prior to administration. In some embodiments, the immune modulator induces T regulatory cells proliferation or induces T regulatory cell production of leukemia inhibitory factor. In some embodiments, the immune modulator is IL-4, IL-10, IL-13, IL-20, TGF-beta, CXCL12, VEGF, PGE-2, or inhibin, or a combination thereof. In some embodiments, the mesenchymal stem cells express STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, or THY-1, or a combination thereof. In some embodiments, the mesenchymal stem cells do not express substantial levels of HLA-DR, CD117, and CD45. In some embodiments, the mesenchymal stem cells are derived from Wharton's Jelly, bone marrow, peripheral blood, mobilized peripheral blood, endometrium, hair follicle, deciduous tooth, testicle, adipose tissue, skin, amniotic fluid, cord blood, omentum, muscle, amniotic membrane, periventricular fluid, placental tissue, pluripotent stem cells, embryonic stem cells, inducible pluripotent stem cells, parthenogenic stem cells, or somatic cell nuclear transfer derived stem cells.

In some embodiments, the embryonic stem cells express stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, or human telomerase reverse transcriptase (hTERT). In some embodiments, the inducible pluripotent stem cells express CD10, CD13, CD44, CD73, CD90, PDGFr-alpha, PD-L2, or HLA-A,B,C and wherein said inducible pluripotent stem cells undergo at least 40 doublings in culture, while maintaining a normal karyotype upon passaging. In some embodiments, the parthenogenic stem cells are generated by addition of a calcium flux inducing agent to activate an oocyte followed by enrichment of cells expressing SSEA-4, TRA 1-60, orTRA 1-81. In some embodiments, the somatic cell nuclear transfer derived stem cells possess a phenotype negative for SSEA-1 and positive for SSEA-3, SSEA-4, TRA-1-60, TRA-1-81, or alkaline phosphatase. In some embodiments, the mesenchymal stem cells are differentiated from a pluripotent stem cell source through culture in the presence of an inhibitor of the SMAD-2/3 pathway or an inhibitor nucleic acid targeting the SMAD-2/3 pathway. In some embodiments, the inhibitor is SB-431542, an antisense oligonucleotide, a hairpin loop short interfering RNA, a chemically synthesized short interfering RNA molecule, or a hammerhead ribozyme. In some embodiments, the mesenchymal stem cells inhibit hepatic stellate cell activation, inhibit hepatic fibrosis, stimulate hepatic regeneration, or augment hepatic oval cell or liver progenitor cell activity.

### Example: Treatment of Carbon Tetrachloride Induced Liver Failure by Treg Cells

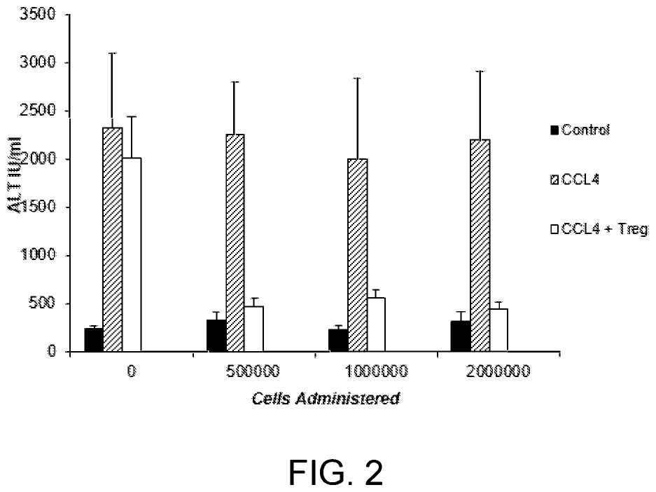

Cryopreserved cord blood bags (1 unit bags) where thawed and washed in CliniMACS buffer (Miltenyi Biotec, Bergish Gladbach, Germany) containing 0.5% HSA (Baxter Healthcare, Westlake Village, Calif.) in order to purify mononuclear cells. Subsequently, cells CD250+ cell enrichment was performed by positive selection using magnetic activated cell sorting (MACS) according to manufacturer's instructions (Miltenyi Biotec, Bergish Gladbach, Germany). Cells were check for viability and subsequently stimulated by co-cultured with CD3/28 co-expressing Dynabeads® (C1inExVivo™ CD3/CD28, Invitrogen Dynal AS, Oslo, Norway) at a 1 cell: 3 bead ratio9 and re-suspended at 1×106 cells/ml in X-VIVO 15 medium (Cambrex BioScience, Walkersville, Md.) supplemented with 10% human AB serum (Gemini Bio-Products, Sacramento, Calif.), 2 mM L-glutamine (Sigma, St. Louis, Mo.), 1% Penicillin-Streptomycin (Gibco/Invitrogen, Grand Island, N.Y.)] and 200 IU/ml interleukin (IL)-2 (CHIRON Corporation, Emeryville, Calif.). Ex vivo co-culture of the CD25+ cells and beads was performed in tissue culture flasks at 37° C. in a 5% CO2-in-air atmosphere. The CB-derived CD25+ enriched T-cells were maintained at 1×106 cells/ml by the addition of fresh medium and IL-2 (maintaining 200 IU/ml) every 48-72 hours. Addition of Wharton Jelly MSC was performed in some cultures. Said MSC where pre-plated at 50% confluency prior to addition of cord blood cells as described above. Cultured Treg cells where purified using CD25 beads and injected intravenously into mice at the indicated concentrations. Serum samples were collected from mice of normal control, mice treated with Carbon Tetrachloride and carbon tetrachloride with cells injected intravenously at 3 days post CCL4 administration. Treg significantly reduced serum levels of ALT Bar graphs represent mean ±SEM of three separate experiments, as shown in FIG. 2. Data shown are representative of three separate experiments performed, with 0 mice per group.

Some aspects described herein further relate to the following numbered embodiments.

1. A method of treating liver failure through administration of a T regulatory cell population, wherein said T regulatory cell population is activated to possess enhanced production of liver regenerating factors by culture with allogeneic mesenchymal stem cells.

2. The method of embodiment 1, wherein said T regulatory cell is or has been rendered activated to induce liver proliferation by enhanced ability to produce hepatocyte growth factor, and to enhance oval cell proliferation.

3. The method of embodiment 1, wherein said placental derived mesenchymal stem cells are in a mitotically inactivated state.

4. The method of embodiment 3, wherein said T regulatory cells are treated with an immune modulator prior to administration.

5. The method of embodiment 1, wherein said placental mesenchymal stem cells are cultured with an immune modulator prior to administration.

6. The method of embodiment 5, wherein said culture with said immune modulator is of time course sufficient to induce ability of T regulatory cells to proliferate.

7. The method of embodiment 5, wherein said culture with said immune modulator is of time course sufficient to induce ability to induce leukemia inhibitory factor production from a T regulatory cell.

8. The method of embodiment 4, wherein said immune modulator is selected from a group consisting of: IL-4, IL-10, IL-13, IL-20, TGF-beta, CXCL12, and inhibin.

9. The method of embodiment 8, wherein said immune modulator is TGF-beta.

10. The method of embodiment 4, wherein said immune modulator is a combination of TGF-beta, VEGF, and PGE-2.

11. The method of embodiment 1, wherein said T regulatory cells are cocultured with cells selected from a group of cells consisting of: a) mesenchymal stem cells; b) T regulatory cells; c) type 2 monocytes; d) CD5 positive B cells; e) type 2 NKT cells; f) tolerogenic dendritic cells; g) gamma delta T cells; h) T cells with immune regulatory properties; i) CD34 cells; j) very small embryonic like stem cells and k) Sertoli cells.

12. The method of embodiment 11, wherein said mesenchymal stem cell is derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) placental tissue.

13. The method of embodiment 12, wherein said mesenchymal stem cells express a marker or plurality of markers selected from a group consisting of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

14. The method of embodiment 13, wherein said mesenchymal stem cells do not express substantial levels of HLA-DR, CD117, and CD45.

15. The method of embodiment 11, wherein said mesenchymal stem cells are generated from a pluripotent stem cell.

16. The method of embodiment 15, wherein said pluripotent stem cell is selected from a group consisting of: a) an embryonic stem cell; b) an inducible pluripotent stem cell; c) a parthenogenic stem cell; and d) a somatic cell nuclear transfer derived stem cell.

17. The method of embodiment 16, wherein said embryonic stem cell population expresses genes selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

18. The method of embodiment 16, wherein said inducible pluripotent stem cell possesses markers selected from a group consisting of: CD10, CD13, CD44, CD73, CD90, PDGFr-alpha, PD-L2, and HLA-A,B,C and possesses ability to undergo at least 40 doublings in culture, while maintaining a normal karyotype upon passaging.

19. The method of embodiment 16, wherein said parthenogenic stem cells wherein said parthenogenically derived stem cells are generated by addition of a calcium flux inducing agent to activate an oocyte followed by enrichment of cells expressing markers selected from a group consisting of SSEA-4, TRA 1-60 and TRA 1-81.

20. The method of embodiment 16, wherein said somatic cell nuclear transfer derived stem cells possess a phenotype negative for SSEA-1 and positive for SSEA-3, SSEA-4, TRA-1-60, TRA-1-81, and alkaline phosphatase.

21. The method of embodiment 15, wherein said mesenchymal stem cells are differentiated from a pluripotent stem cell source through culture in the presence of an inhibitor of the SMAD-2/3 pathway.

22. The method of embodiment 21, wherein said mesenchymal stem cells are differentiated from a pluripotent stem cell source through culture in the presence of an inhibitor nucleic acid targeting the SMAD-2/3 pathway.

23. The method of embodiment 22, wherein said nucleic acid inhibitor is selected from a group consisting of: a) an antisense oligonucleotide; b) a hairpin loop short interfering RNA; c) a chemically synthesized short interfering RNA molecule; and d) a hammerhead ribozyme.

24. The method of embodiment 22, wherein said inhibitor of the SMAD-2/3 pathway is a small molecule inhibitor.

25. The method of embodiment 24, wherein said small molecule inhibitor is SB-431542.

26. The method of embodiment 15, wherein a selection process is used to enrich for mesenchymal stem cells differentiated from said pluripotent stem cell population.

27. The method of embodiment 26, wherein said enrichment method comprises of positively selecting for cells expressing a marker associated with mesenchymal stem cells.

28. The method of embodiment 27, wherein said marker of mesenchymal stem cells is selected from a group consisting of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

29. The method of embodiment 11, wherein said mesenchymal stem cell is endowed with augmented immune modulatory activity, said augmentation induced by exposure to an agent or plurality of agents inducing a stress response in said mesenchymal stem cell.

30. The method of embodiment 29, wherein said immune modulatory activity is ability to inhibit hepatic stellate cell activation.

31. The method of embodiment 29, wherein said immune modulatory activity is ability to inhibit hepatic fibrosis.

32. The method of embodiment 29, wherein said immune modulatory activity is ability to stimulate hepatic regeneration.

33. The method of embodiment 29, wherein said immune modulatory activity is ability to augment oval cell or liver progenitor cell activity.

34. The method of embodiment 30, wherein said inhibition of stellate cell activity is associated with reduction in liver fibrosis.

35. The method of embodiment 32, wherein said liver fibrosis is defined as reduction in functional hepatocytes.

36. The method of embodiment 32, wherein said hepatic regeneration is defined as stimulation of the initiation or proliferation phase of liver tissue growth after injury.

37. The method of embodiment 32, wherein said liver regeneration is defined as proliferation of hepatocytes.

38. The method of embodiment 33, wherein said oval cell or liver progenitor cell is defined as a cell responsible for generation of new hepatic tissue under conditions where hepatocytes stop proliferating in response to injury or damage.

39. The method of embodiment 1, wherein said T regulatory cells are derived from peripheral blood mononuclear cells.

40. The method of embodiment 1, wherein said T regulatory cells are derived from mobilized peripheral blood mononuclear cells.

41. The method of embodiment 40, wherein said mobilization is achieved by administration of G-CSF.

42. The method of embodiment 40, wherein said mobilization is achieved by administration of flt-3 ligand.

43. The method of embodiment 40, wherein said mobilization is achieved by administration of thrombopoieting.

44. The method of embodiment 40, wherein said mobilization is achieved by administration of Mozobil.

45. The method of embodiment 1, wherein said T regulatory cells are derived from cord blood.

46. The method of embodiment 1, wherein said T regulatory cells are derived from menstrual blood.

47. The method of embodiment 1, wherein said T regulatory cells are derived from adipose stromal vascular fraction cells.

48. The method of embodiment 1, wherein said T regulatory cells possess ability to inhibit proliferation of naive T cells stimulated with a signal that activates proliferation.

49. The method of embodiment 48, wherein said signal that activates proliferation is selected from a group consisting of: a) anti-CD3 and anti-CD28 beads; b) concanavalin A; c) PHA; and d) stimulation with alloreactive antigen presentation cells.

50. The method of embodiment 1, wherein said T regulatory cell is capable of suppressing maturation of dendritic cells.

51. The method of embodiment 50, wherein said maturation is dendritic cells is upregulation of molecules selected from a group consisting of: CD80; CD40; CD86; and HLA II.

52. The method of embodiment 1, wherein said T regulatory cell possesses expression of GITR ligand.

53. The method of embodiment 1, wherein said T regulatory cell expresses neuropilin-1.

54. The method of embodiment 1, wherein said T regulatory cell expresses CTLA-4.

55. The method of embodiment 1, wherein said T regulatory cell expresses CD25.

56. The method of embodiment 1, wherein said T regulatory cell expresses CD105.

57. The method of embodiment 1, wherein said T regulatory cell expresses membrane bound TGF-beta.

58. The method of embodiment 1, wherein said T regulatory cell produces IL-10.

59. The method of embodiment 1, wherein said T regulatory cells are activated by exposure to vasoactive intestinal peptide.

60. The method of embodiment 1, wherein said T regulatory cells are activated by exposure to IL-10.

61. The method of embodiment 1, wherein said T regulatory cells are activated by exposure to TGF-beta.

62. The method of embodiment 1, wherein said T regulatory cells are activated by exposure to mesenchymal stem cell conditioned media.

63. The method of embodiment 1, wherein said T regulatory cells are activated by exposure to mesenchymal stem cell derived exosomes.

64. The method of embodiment 1, wherein said T regulatory cells are activated by exposure to BDNF.

65. The method of embodiment 1, wherein said T regulatory cells are activated by exposure to human chorionic gonadotropin.

66. The method of embodiment 1, wherein said T regulatory cells are activated by exposure to VEGF.

67. The method of embodiment 1, wherein said T regulatory cells are activated by exposure to CD3 and CD28 antibodies.

68. The method of embodiment 1, wherein said T regulatory cells are anergic T cells.

69. The method of embodiment 1, wherein said T regulatory cells are activated by exposure to hypoxic conditions.

70. The method of embodiment 1, wherein said T regulatory cells are activated by exposure to angiopoietin.

71. The method of embodiment 1, wherein said T regulatory cells express FoxP3.

72. The method of embodiment 1, wherein said T regulatory cells express CD39.

73. The method of embodiment 1, wherein said T regulatory cells express CD73.

74. The method of embodiment 1, wherein said T regulatory cells express CD127.

75. The method of embodiment 1, wherein said T regulatory cells express GARP.

76. The method of embodiment 1, wherein said T regulatory cells are activated by exposure to rapamycin.

It is to be understood that the description, specific examples and data, while indicating exemplary embodiments, are given by way of illustration and are not intended to limit the various embodiments of the present disclosure. Various changes and modifications within the present disclosure will become apparent to the skilled artisan from the description and data contained herein, and thus are considered part of the various embodiments of this disclosure.

1. A pharmaceutical composition for treating a liver disorder comprising activated T regulatory cells, wherein the composition is formulated for administration to a subject having a liver disorder.

2. The pharmaceutical composition of claim 1, wherein the activated T regulatory cells are allogeneic T regulatory cells.

3. The pharmaceutical composition of claim 1, wherein the activated T regulatory cells produce hepatocyte growth factor.

4. The pharmaceutical composition of claim 1, wherein the activated T regulatory cells enhance hepatic oval cell production.

5. The pharmaceutical composition of claim 1, wherein the composition is formulated for intravenous administration.

6. The pharmaceutical composition of claim 1, wherein the activated T regulatory cells are present in an amount of about 5×10⁵ to 2×10⁶ cells.

7. The pharmaceutical composition of claim 1, wherein the activated T regulatory cells are present in an amount of about 1×10⁶ cells per milliliter.

8. The pharmaceutical composition of claim 1, wherein the composition decreases a subject's serum alanine aminotransferase (ALT), aspartate aminotransferase (AST), albumin, or bilirubin levels to 5-20%, 10-30%, 20-40%, 30-50%, 40-60%, or 50-70% that of a healthy subject.

9. The pharmaceutical composition of claim 1, wherein the liver disorder is hepatitis, cirrhosis, chronic liver disease, acute liver disease, or liver failure.

10. The pharmaceutical composition of claim 1, further comprising a compound for treating liver disease.

11. The pharmaceutical composition of claim 1, further comprising a pharmaceutically acceptable carrier.

12. (canceled)

13. A method of treating liver failure comprising administering a composition comprising activated T regulatory cells, wherein the T regulatory cells are activated to possess enhanced production of liver regenerating factors by culture with allogeneic mesenchymal stem cells.

14. The method of claim 13, wherein said population of T regulatory cells is or has been rendered activated to induce liver proliferation by enhanced ability to produce hepatocyte growth factor, and to enhance hepatic oval cell proliferation.

15. The method of claim 13, wherein said mesenchymal stem cells are in a mitotically inactivated state.

16. The method of claim 13, wherein said T regulatory cells or said mesenchymal stem cells are treated with an immune modulator prior to administration.

17. The method of claim 16, wherein said culture with said immune modulator induces T regulatory cells proliferation or induces T regulatory cell production of leukemia inhibitory factor.

18. The method of claim 16, wherein said immune modulator is IL-4, IL-10, IL-13, IL-20, TGF-beta, CXCL12, VEGF, PGE-2, or inhibin, or a combination thereof.

19. The method of claim 13, wherein said T regulatory cells are cocultured with type 2 monocytes, CD5 positive B cells, type 2 NKT cells, tolerogenic dendritic cells, gamma delta T cells, T cells with immune regulatory properties, CD34 cells, very small embryonic like stem cells, or Sertoli cells.

20. The method of claim 13, wherein treating liver failure comprises reducing liver fibrosis or stimulating proliferation of liver tissue growth after injury.

21. (canceled)

22. The method of claim 13, wherein said T regulatory cells are derived from peripheral blood mononuclear cells, mobilized peripheral blood mononuclear cells, cord blood, menstrual blood, or adipose stromal vascular fraction cells.

23. The method of claim 13, wherein said activated T regulatory cells inhibit proliferation of naive T cells stimulated with a signal that activates proliferation.

24. The method of claim 13, wherein said activated T regulatory cells suppress maturation of dendritic cells.

25. The method of claim 13, wherein said activated T regulatory cells express neuropilin-1, CTLA-4, CD25, CD39, CD73, CD105, CD127, FoxP3, GARP, GITR ligand, IL-10, or membrane bound TGF-beta.

26. The method of claim 13, wherein said activated T regulatory cells are activated by exposure to vasoactive intestinal peptide, IL-10, TGF-beta, mesenchymal stem cell conditioned media, mesenchymal stem cell derived exosomes, BDNF, human chorionic gonadotropin, VEGF, CD3 or CD28 antibodies, hypoxic conditions, rapamycin, or angiopoietin.
